(19) United States
(12) Patent Application Publication
(10) Pub. No.: US 2021/0322452 A1
(43) Pub. Date: Oct. 21, 2021

(54) COGNITIVE FUNCTION IMPROVING COMPOSITION COMPRISING NOVEL POST FERMENTED TEA-DERIVED KAEMPFEROL-BASED COMPOUND

(71) Applicant: AMOREPACIFIC CORPORATION, Seoul (KR)

(72) Inventors: Yong-Deog HONG, Yongin-si, Gyeonggi-do (KR); Minsik CHOI, Yongin-si, Gyeonggi-do (KR); Si Young CHO, Yongin-si, Gyeonggi-do (KR); Jeong-Kee KIM, Yongin-si, Gyeonggi-do (KR)

(21) Appl. No.: 16/340,753

(22) PCT Filed: Oct. 16, 2017
(86) PCT No.: PCT/KR2017/011401

§ 371 Date: Apr. 10, 2019

(30) Foreign Application Priority Data
Oct. 18, 2016 (KR) 10-2016-0135302
Sep. 18, 2017 (KR) 10-2017-0119273

(51) Int. Cl. A61K 31/7048; A61K 36/82; A61P 25/28; A23L 33/00; A23L 33/105; A23L 2/52

(52) U.S. Cl. CPC A61K 31/7048; A61K 36/82; A61P 25/28; A23L 33/40; A23L 33/105; A23V 2002/00; A61K 2236/331; A61K 2236/333; A61K 2236/37; A61K 2236/39; A23L 2/52

(57) ABSTRACT

The present specification relates to a cognitive function decrease improving composition comprising a novel compound separated from post fermented tea, an isomer thereof, a pharmaceutically acceptable salt thereof, a hydrate thereof, or a solvate thereof, the compound being capable of being widely used in fields related to cognitive functions and nerve cell protection.

### TECHNICAL FIELD

The present specification relates to a cognitive function improving composition comprising a novel kaempferol-based compound.

### BACKGROUND ART

As the improvement in standards of living and the development in the medical industry have enabled the treatment of intractable diseases including cancer, the life span of humans has increased. However, the resultant aging of the population has caused a cognitive function decrease and an increase in chronic degenerative neurologic diseases, and this rather relatively drops the quality of life. Nerve cell dysfunction and damage may be caused by specific proteins that are susceptible to aggregation, and a number of neurologic diseases are characterized by such conditions. Such neurologic diseases include diseases such as Alzheimer's disease.

As the elderly population has increased, the need for treatment and prevention of aging, cognitive function decrease, degenerative neurologic diseases, and brain diseases has increased. Hence, researches on the prevention, treatment, alleviation, and improvement of such aging and diseases have been steadily conducted, but the existing substances have problems that the effects thereof are unclear or side effects are caused. It is thus required to develop therapeutic agents derived from natural products to solve these problems.

Green tea is consumed as a coarse tea in the leaf form or as a fermented tea to feel deeper flavor. Fermented green tea means one obtained by subjecting green tea leaves to oxidation treatment and includes fermented tea oxidized using oxidizing enzymes present in the tea leaves and post fermented tea fermented using microorganisms other than the enzymes present in the tea leaves. Fermented green tea can be divided into weak fermented tea, semi-fermented tea, and fully fermented tea depending on the degree of fermentation. For example, fermented green tea is called various names such as green tea, oolong tea, black tea, and pure tea depending on the type and degree of fermentation.

Fermented tea may have not only a difference in flavor compared to coarse tea but also a great difference in the kind and content of active ingredient depending on the specific fermentation process and the kind of microorganisms. Since various compounds can be produced and separated from green tea as described above, a variety of attempts have been made to separate and identify unknown novel compounds using green tea.

### SUMMARY OF INVENTION

### Technical Problem

In an aspect, an object of the present invention is to discover a novel post fermented tea-derived compound and to use this for cognitive function improvement and nerve cell protection.

### Solution to Problem

In an aspect, the present invention provides a cognitive function decrease improving composition containing a compound represented by the following Chemical Formula 1, an isomer thereof, a pharmaceutically acceptable salt thereof, a hydrate thereof, a solvate thereof, or a post fermented tea extract containing this as an active ingredient.

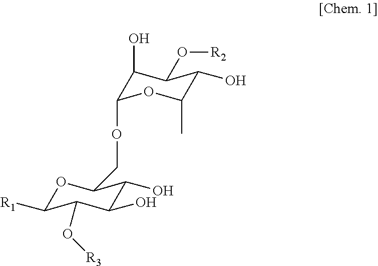

where R₁ may represent C₁₅H₉O₆, R₂ may represent C₆H₁₁O₅, and R₃ may represent C₉H₇O₂.

In another aspect, the present invention also provides a nerve cell protecting or neurologic disease treating composition containing the compound, an isomer thereof, a pharmaceutically acceptable salt thereof, a hydrate thereof, a solvate thereof, or a post fermented tea extract containing this as an active ingredient.

In another aspect, the present invention also provides a method for improving cognitive function decrease, a method for treating cognitive function decrease, a method for protecting a nerve cell, or a method for treating a neurologic disease, which includes administering an effective amount of the compound, an isomer thereof, a pharmaceutically acceptable salt thereof, a hydrate thereof, a solvate thereof, or a post fermented tea extract containing this to an individual in need thereof.

In another aspect, the present invention also provides use of the compound, an isomer thereof, a pharmaceutically acceptable salt thereof, a hydrate thereof, a solvate thereof, or a post fermented tea extract containing this for the preparation of a cognitive function decrease improving, cognitive function decrease treating, nerve cell protecting, or neurologic disease treating composition.

In another aspect, the present invention also provides the compound, an isomer thereof, a pharmaceutically acceptable salt thereof, a hydrate thereof, a solvate thereof, or a post fermented tea extract containing this as an active ingredient to be used for the cognitive function decrease improvement, cognitive function decrease treatment, nerve cell protection, and neurologic disease treatment.

In still another aspect, the present invention also provides non-therapeutic use of the compound, an isomer thereof, a pharmaceutically acceptable salt thereof, a hydrate thereof, a solvate thereof, or a post fermented tea extract containing this as an active ingredient for cognitive function decrease improvement and nerve cell protection.

### Advantageous Effects of Invention

In an aspect of the present invention, by using a novel compound separated from post fermented tea in the fields of cognitive function improvement and nerve cell protection, the novel compound can be widely used in the post fermented tea-related industries, the cognitive function field, and the neuroscience field.

### BRIEF DESCRIPTION OF DRAWINGS

FIG. 1 illustrates the MS spectrum of a compound according to an aspect of the present invention.

FIG. 2 illustrates the ¹H-NMR (nuclear magnetic resonance) spectrum of a compound according to an aspect of the present invention.

FIG. 3 illustrates the ¹³C-NMR spectrum of a compound according to an aspect of the present invention.

FIG. 4 illustrates the ¹H-¹³C HSQC (heteronuclear single quantum coherence) spectrum of a compound according to an aspect of the present invention.

FIG. 5 illustrates the ¹H-¹³C HMBC (heteronuclear multiple-bond coherence) spectrum of a compound according to an aspect of the present invention.

FIG. 6 illustrates the influence of a compound according to an aspect of the present invention on beta amyloid aggregation.

### DESCRIPTION OF EMBODIMENTS

In the present specification, “post fermentation” includes fermentation using microorganisms or a substance other than the enzymes present in tea leaves. Post fermented tea includes the green tea fermented by the above-mentioned method.

In the present specification, “extract” includes all substances obtained by extracting components contained in natural products from the natural products regardless of the extraction method or the kind of component. “Extract” is a broad concept including all those obtained by extracting components dissolving in a solvent from natural products using water or an organic solvent, those obtained by extracting only specific components of natural products, such as oil, and fractions obtained by fractionating those thus obtained again using a specific solvent and the like, for example.

In the present specification, “fraction” includes fractions obtained by fractionating a specific substance or extract using a certain solvent, remnants, and those obtained by extracting these again using a specific solvent. The fractionation method and the extraction method may be any methods known to those skilled in the art.

In the present specification, “isomers” particularly includes not only optical isomers (e.g., essentially pure enantiomers, essentially pure diastereomers, or mixtures thereof) but also conformation isomers (i.e., isomers different only in the angle of one or more chemical bonds), position isomers (particularly tautomers), or geometric isomers (e.g., cis-trans isomers).

In the present specification, “essentially pure” means that a specific compound having enantiomers or diastereomers is present at about 90% or more, preferably about 95% or more, more preferably about 97% or more or about 98% or more, even more preferably about 99% or more, yet more preferably about 99.5% or more (w/w) in the case of being used in connection with, for example, enantiomers or diastereomers.

In the present specification, “pharmaceutically acceptable” means it is recognized that one can be used for an animal, more specifically humans by avoiding significant toxic effects when being used in conventional medicinal dosages as being capable of being approved or as being approved by the government or a regulatory agency equivalent thereto or as being enumerated in the pharmacopeia or as being described in other general pharmacopeias.

In the present specification, “pharmaceutically acceptable salt” means a salt according to an aspect of the present invention which is pharmaceutically acceptable and has the desired pharmacological activity of the parent compound. The salt may include (1) acid addition salts formed using inorganic acids such as hydrochloric acid, hydrobromic acid, sulfuric acid, nitric acid, and phosphoric acid; or organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2,2,2]-oct-2-en-1-carboxylic acid, glucoheptonic acid, 3-phenylpropionic acid, trimethylacetic acid, tert-butylacetic acid, laurylsulfuric acid, gluconic acid, glutamic acid, hydroxynaphthoic acid, salicylic acid, stearic acid, and muconic acid; or (2) a salt formed when an acidic proton present in a parent compound is substituted.

In the present specification, “hydrate” means a compound bonded with water and is a broad concept that includes an inclusion compound in which water and the compound do not have chemical bonding force therebetween.

In the present specification, “solvate” means a higher order compound formed between a molecule or ion of a solute and a molecule or ion of a solvent.

In an aspect, the present invention provides a cognitive function decrease improving composition containing a compound represented by the following Chemical Formula 1, an isomer thereof, a pharmaceutically acceptable salt thereof, a hydrate thereof, a solvate thereof, or a post fermented tea extract containing this as an active ingredient.

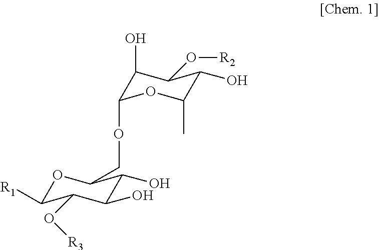

where R₁ may represent C₁₅H₉O₆, R₂ may represent C₆H₁₁O₅, and R₃ may represent C₉H₇O₂.

According to an embodiment, R₁ may be a compound represented by the following Chemical Formula 2.

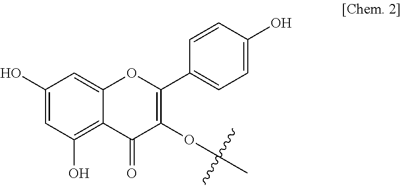 [SMILES: *Oc1c(-c2ccc(O)cc2)oc2cc(O)cc(O)c2c1=O]

According to another embodiment, R₂ may be a compound represented by the following Chemical Formula 3.

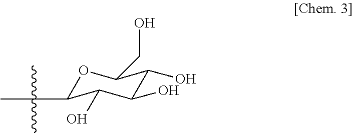 [SMILES: *C1OC(CO)C(O)C(O)C1O]

R₃ may be a compound represented by the following Chemical Formula 4.

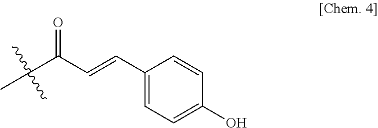 [SMILES: *C(=O)/C=C/c1ccc(O)cc1]

According to another embodiment, the compound may be kaempferol3-O-[2-O″-(E)-p-coumaroyl][beta-D-glucopyranosyl-(1→3)-O-alpha-L-rhamnopyranosyl-(1→6)-O-beta-D-glucopyranoside]. The compound can be represented by the following Chemical Formula 5.

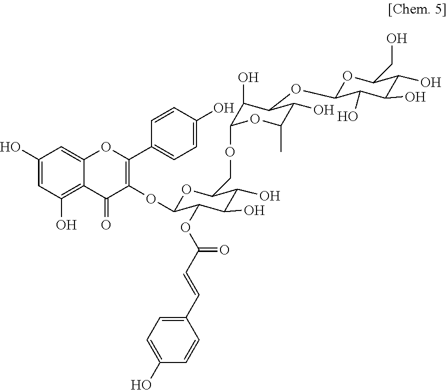 [SMILES: CC1OC(OCC2OC(Oc3c(-c4ccc(O)cc4)oc4cc(O)cc(O)c4c3=O)C(OC(=O)/C=C/c3ccc(O)cc3)C(O)C2O)C(O)C(OC2OC(CO)C(O)C(O)C2O)C1O]

According to an embodiment of the present invention, the method for manufacturing the compound, an isomer thereof, a pharmaceutically acceptable salt thereof, a hydrate thereof, or a solvate thereof may include synthesis, separation from natural products, and the like.

According to another embodiment, the post fermentation may be conducted by strain inoculation. The strain may be a strain selected from Saccharomyces sp., Bacillus sp. Lactobacillus sp., or Leuconostoc mesenteroides sp. and may be preferably selected from Saccharomyces cerevisiae, Lactobacillus casei, Bacillus subtilis, Lactobacillus bulgarius, or Leuconostoc mesenteroides. According to still another embodiment, the post fermented tea may be a post fermented green tea.

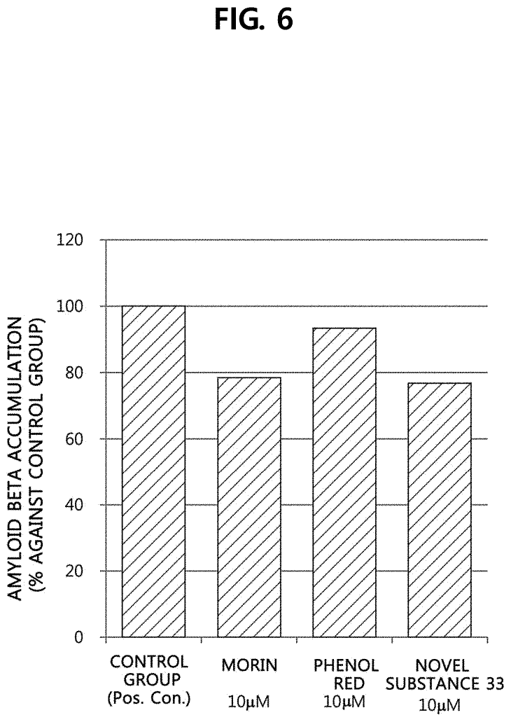

In an aspect of the present invention, the compound is a compound which has been discovered as a result of continuous researches on post fermented tea by the present inventors. The β-amyloid aggregation assay has been conducted using the compound, and as a result, it has been confirmed that the compound exhibits an effect of inhibiting β-amyloid aggregation and β-amyloid plaque formation superior to those of morin and phenol red which are known inhibitors. Consequently, it has been revealed that the compound according to an aspect of the present invention can be used to prevent, treat, and improve cognitive function decrease associated with β-amyloid, and it has also been demonstrated that the compound can be used to protect nerve cells from damage and death due to β-amyloid aggregation (see FIG. 6).

In addition, in an aspect of the present invention, the compound enhances BDNF expression and reduces DNMT1 expression in nerve cells. In other words, it has been revealed that the present invention can be helpfully utilized for the prevention and treatment of degenerative neurologic diseases such as cognitive function decrease, dementia, and Alzheimer's disease associated with reduced expression of BDNF or enhanced expression of DNMT1.

In an aspect, the present invention may also be a method for improving cognitive function decrease, a method for treating cognitive function decrease, a method for protecting a nerve cell, or a method for treating a neurologic disease, which includes administering an effective amount of the compound, an isomer thereof, a pharmaceutically acceptable salt thereof, a hydrate thereof, a solvate thereof, or a post fermented tea extract containing this to an individual in need thereof.

In another aspect, the present invention may also relate to the use of the compound, an isomer thereof, a pharmaceutically acceptable salt thereof, a hydrate thereof, a solvate thereof, or a post fermented tea extract containing this for the preparation of a cognitive function decrease improving, cognitive function decrease treating, nerve cell protecting, or neurologic disease treating composition.

In another aspect, the present invention may also be the compound, an isomer thereof, a pharmaceutically acceptable salt thereof, a hydrate thereof, a solvate thereof, or a post fermented tea extract containing this as an active ingredient to be used for the cognitive function decrease improvement, cognitive function decrease treatment, nerve cell protection, and neurologic disease treatment.

In still another aspect, the present invention may also relate to non-therapeutic use of the compound, an isomer thereof, a pharmaceutically acceptable salt thereof, a hydrate thereof, a solvate thereof, or a post fermented tea extract containing this as an active ingredient for cognitive function decrease improvement and nerve cell protection.

In an embodiment, the extraction may be extraction using one or more solvents selected from water, hot water, a C₁ to C₆ lower alcohol, or any mixed solvent thereof. According to another embodiment, the lower alcohol may be any single alcohol which can be commonly used in the art or a mixture containing the alcohol, and the lower alcohol may be preferably ethanol.

According to another aspect of the present invention, the extract may be a fraction which is fractionated using a ketone after extraction.

According to another embodiment, the ketone may include acetone, carvone, pulegone, isolongifolanone, 2-heptanone, 2-pentanone, 3-hexanone, 3-heptanone, 4-heptanone, 2-octanone, 3-octanone, 2-nonanone, 3-nonanone, 2-undecanone, 2-tridecanone, methyl isopropyl ketone, ethyl isoamyl ketone, butylidene acetone, methyl heptenone, dimethyl octenone, geranyl acetone, farnesyl acetone, 2,3-pentadione, 2,3-hexadione, 3,4-hexadione, 2,3-heptadione, amyl cyclopentanone, amyl cyclopentenone, 2-cyclopentyl cyclopentanone, hexyl cyclopentanone, 2-n-heptyl cyclopentanone, cis-jasmone, dihydrojasmone, methyl corylone, 2-tert-butyl cyclohexanone, p-tert-butyl cyclohexanone, 2-sec-butyl cyclohexanone, celery ketone, krypton, p-tert-pentyl cyclohexanone, methyl cyclocitrone, nerone, 4-cyclohexyl-4-methyl-2-pentanone, oxide ketone, emoxyfurone, methylnaphthyl ketone, α-methyl anisalacetone, anisyl acetone, p-methoxyphenyl acetone, benzylidene acetone, p-methoxyacetophenone, p-methylacetophenone, propiophenone, acetophenone, α-dynascone, iritone, ionone, pseudoionone, methyl ionone, methyl iritone, 2,4-di-tert-butyl cyclohexanone, allyl ionone, 2-acetyl-3,3-dimethyl norbornane, verbenone, fenchone, cyclopentadecanone, cyclohexadecenone, and the like. The ketone may include all of ketones as solvents to be commonly used in the art and mixtures thereof, and the ketone may be preferably acetone.

According to an aspect of the present invention, the content of the compound represented by Chemical Formula 1, an isomer thereof, a pharmaceutically acceptable salt thereof, a hydrate thereof, or a solvate thereof in the composition may be from 0.00001 wt % to 10 wt % based on the total weight of the composition. The content may be 0.00001 wt % or more, 0.00005 wt % or more, 0.0001 wt % or more, 0.0005 wt % or more, 0.001 wt % or more, 0.005 wt % or more, 0.01 wt % or more, 0.05 wt % or more, 0.1 wt % or more, 0.5 wt % or more, 1 wt % or more, 2 wt % or more, 3 wt % or more, 4 wt % or more, 5 wt % or more, 6 wt % or more, 7 wt % or more, 8 wt % or more, or 9 wt % or more based on the total weight of the composition. In addition, the content may be 10 wt % or less, 9 wt % or less, 8 wt % or less, 7 wt % or less, 6 wt % or less, 5 wt % or less, 4 wt % or less, 3 wt % or less, 2% or less, 1% or less, 0.5% or less, 0.1% or less, 0.05% or less, 0.01% or less, 0.005 wt % or less, 0.001 wt % or less, 0.0005 wt % or less, 0.0001 wt % or less, 0.00005 wt % or less, or 0.00003 wt % or less based on the total weight of the composition.

According to another aspect of the present invention, the content of the post fermented tea extract in the composition may be from 0.1 wt % to 90 wt % based on the total weight of the composition. The content may be 0.1 wt % or more, 1 wt % or more, 5 wt % or more, 10 wt % or more, 15 wt % or more, 20 wt % or more, 25 wt % or more, 30 wt % or more, 35 wt % or more, 40 wt % or more, 45 wt % or more, 50 wt % or more, 55 wt % or more, 60 wt % or more, 65 wt % or more, 70 wt % or more, 75 wt % or more, 80 wt % or more, or 85 wt % or more based on the total weight of the composition. In addition, the content may be 90 wt % or less, 85 wt % or less, 80 wt % or less, 75 wt % or less, 70 wt % or less, 65 wt % or less, 60 wt % or less, 55 wt % or less, 50 wt % or less, 45 wt % or less, 40 wt % or less, 35 wt % or less, 30 wt % or less, 25 wt % or less, 20 wt % or less, 15 wt % or less, 10 wt % or less, 5 wt % or less, 1 wt % or less, or 0.5 wt % or less based on the total weight of the composition.

According to still another aspect of the present invention, the extract may contain the compound represented by Chemical Formula 1, an isomer thereof, a pharmaceutically acceptable salt thereof, a hydrate thereof, or a solvate thereof at 0.0001 wt % or more, 0.0005 wt % or more, 0.001 wt % or more, 0.005 wt % or more, 0.01 wt % or more, 0.05 wt % or more, 0.1 wt % or more, 0.5 wt % or more, 1 wt % or more, 3 wt % or more, 5 wt % or more, 7 wt % or more, 10 wt % or more, 12 wt % or more, 15 wt % or more, or 18 wt % or more based on the total weight of the extract. In addition, the extract may contain the compound represented by Chemical Formula 1, an isomer thereof, a pharmaceutically acceptable salt thereof, a hydrate thereof, or a solvate thereof at 20 wt % or less, 15 wt % or less, 12 wt % or less, 10 wt % or less, 7 wt % or less, 5 wt % or less, 3 wt % or less, 1 wt % or less, 0.5 wt % or less, 0.1 wt % or less, 0.05 wt % or less, 0.01 wt % or less, 0.005 wt % or less, 0.001 wt % or less, 0.0005 wt % or less, or 0.0003 wt % or less based on the total weight of the extract. Preferably, the extract may contain the compound represented by Chemical Formula 1, an isomer thereof, a pharmaceutically acceptable salt thereof, a hydrate thereof, or a solvate thereof at from 0.0001 wt % to 20 wt % based on the total weight of the extract.

According to still another aspect of the present invention, the dosage of the compound represented by Chemical Formula 1, an isomer thereof, a pharmaceutically acceptable salt thereof, a hydrate thereof, or a solvate thereof by administration of the composition may be from 0.001 mg/kg/day to 100 mg/kg/day. The dosage may be 0.001 mg/kg/day or more, 0.005 mg/kg/day or more, 0.01 mg/kg/day or more, 0.05 mg/kg/day or more, 0.1 mg/kg/day or more, 0.5 mg/kg/day or more, 1 mg/kg/day or more, 5 mg/kg/day or more, 10 mg/kg/day or more, 15 mg/kg/day or more, 20 mg/kg/day or more, 25 mg/kg/day or more, 30 mg/kg/day or more, 35 mg/kg/day or more, 40 mg/kg/day or more, 45 mg/kg/day or more, 50 mg/kg/day or more, 55 mg/kg/day or more, 60 mg/kg/day or more, 65 mg/kg/day or more, 7 mg/kg/day or more, 75 mg/kg/day or more, 80 mg/kg/day or more, 85 mg/kg/day or more, 90 mg/kg/day or more, or 95 mg/kg/day or more. In addition, the dosage may be 100 mg/kg/day or less, 95 mg/kg/day or less, 90 mg/kg/day or less, 85 mg/kg/day or less, 80 mg/kg/day or less, 75 mg/kg/day or less, 70 mg/kg/day or less, 65 mg/kg/day or less, 60 mg/kg/day or less, 55 mg/kg/day or less, 50 mg/kg/day or less, 45 mg/kg/day or less, 40 mg/kg/day or less, 35 mg/kg/day or less, 30 mg/kg/day or less, 25 mg/kg/day or less, 20 mg/kg/day or less, 15 mg/kg/day or less, 10 mg/kg/day or less, 5 mg/kg/day or less, 1 mg/kg/day or less, 0.5 mg/kg/day or less, 0.1 mg/kg/day or less, 0.05 mg/kg/day or less, 0.01 mg/kg/day or less, 0.005 mg/kg/day or less, 0.003 mg/kg/day or less.

According to an embodiment, the cognitive function decrease may be caused by any one or more selected from the group consisting of aggregation of β-amyloid, plaque formation of β-amyloid, reduced expression of brain-derived neurotrophic factor (BDNF), and enhanced expression of DNMT1 (DNA (cytosine-5)-methyltransferase 1).

According to another embodiment, the cognitive function decrease may include one or more selected from the group consisting of memory loss, cognition decline, discrimination decline, depression, and forgetfulness.

According to still another embodiment, the improvement may be achieved through one or more selected from the group consisting of inhibition of β-amyloid aggregation, inhibition of β-amyloid plaque formation, degradation of β-amyloid plaque or aggregated β-amyloid, enhancement of BDNF expression, and reduction of DNMT1 expression.

According to an aspect of the present invention, the composition may be a nerve cell protecting composition.

According to another aspect, the nerve cell protection may be to protect nerve cells from influence of aggregation or plaque formation of β-amyloid, reduced expression of BDNF, and enhanced expression of DNMT1. It is known that aggregated β-amyloid damages and kills nerve cells, and it is thus possible to protect nerve cells by inhibiting aggregation or plaque formation of β-amyloid according to an aspect of the present invention. In addition, DNMT1 inhibits gene expression by causing DNA methylation, and this thus causes a problem in the BDNF expression and the like and leads to a cognitive ability decrease. In an aspect, the present invention inhibits DNA methyltransferase 1 (DNMT1) activity, thus inhibits DNA methylation, and is effective in the improvement of cognitive ability and degenerative neurologic diseases through nerve cell protection.

According to another aspect of the present invention, the composition may be a pharmaceutical composition or a food composition. In an aspect, the composition may be a pharmaceutical composition for preventing or treating degenerative neurologic diseases. In another aspect, the degenerative neurologic diseases may be caused by one or more selected from the group consisting of aggregation of β-amyloid, reduced expression of BDNF, and enhanced expression of DNMT1. In another aspect, the degenerative neurologic diseases include dementia, Alzheimer's disease, forgetfulness and the like.

The pharmaceutical composition according to an aspect of the present invention may be administered orally, parenterally, rectally, topically, transdermally, intravenously, intramuscularly, intraperitoneally, subcutaneously, and the like. The formulation for oral administration may be tablets, pills, soft and hard capsules, granules, powders, fine granules, liquids, emulsions, or pellets but is not limited thereto. The formulation for parenteral administration may be solutions, suspensions, emulsions, gels, injections, drops, suppositories, patches, or spray agents but is not limited thereto. The formulations can be readily prepared according to conventional methods in the art and may additionally contain surfactants, excipients, wetting agents, emulsifying accelerators, suspending agents, salts or buffers for controlling the osmotic pressure, colorants, spices, stabilizers, antiseptics, preservatives, or other commonly used adjuvants.

The applied amount or dosage of the pharmaceutical composition according to an aspect of the present invention varies depending on the age, sex, weight, pathological condition and severity of the subject to be administered, the administration route, or the judgment of the prescriber. The determination of the applied amount based on these factors is within the level of those skilled in the art.

The formulation of the food composition is not particularly limited but may be formulated into, for example, tablets, granules, pills, powders, liquid preparations such as drinks, caramels, gels, bars, and tea bags. The ingredients commonly used in the field other than the active ingredient may be appropriately chosen and blended in the food composition of each formulation by those skilled in the art depending on the formulation or purpose of use without difficulty. A synergistic effect may be obtained in the case of simultaneously applying the active ingredient and other raw materials.

The composition may be administered by various methods such as simple ingestion, drinking, injection administration, spray administration, or squeeze administration.

In the food composition according to an aspect of the present invention, the determination of the dosage of the active ingredient is within the level of those skilled in the art, and the dosage of the active ingredient may vary depending on various factors such as the age, health condition, and complication of the subject to be administered.

The food composition according to an aspect of the present invention includes, for example, any type of processed food such as various foods such as chewing gums, caramel products, candies, ice creams, and confectioneries and beverages such as soft drinks, mineral water, and alcoholic beverages. The food composition may be a health and functional food containing vitamins and minerals.

In addition to the above, the food composition according to an aspect of the present invention may contain various nutrients, vitamins, minerals (electrolytes), flavors such as synthetic flavors and natural flavors, colorants and enhancers (cheese, chocolate, etc.), pectic acid and its salts, alginic acid and its salts, organic acids, protective colloid thickeners, pH adjusting agents, stabilizers, antiseptics, glycerin, alcohols, carbonating agents used in carbonated drinks and the like. In addition, the food compositions according to an aspect of the present invention may contain natural fruit juice and pulp for the production of fruit juice drinks and vegetable drinks. These ingredients may be used independently or in combination. The proportion of these additives is not so important, but these additives are generally contained in a range of from 0 to about 50 parts by weight per 100 parts by weight of the composition according to an aspect of the present invention.

### EXAMPLES

Hereinafter, the configuration and effects of the present specification will be described in more detail with reference to Examples and Experimental Examples. However, these examples are provided for illustrative purposes only in order to facilitate understanding of the present specification, and the scope and range of the present specification are not limited by the following examples.

### [Example 1] Preparation of Post Fermented Tea Sample

Water was added to green tea made of green tea (Camellia sinensis var. Yabukita) leaves, and the water content was adjusted to 40 wt %. The green tea was inoculated with Bacillus subtillis at 5×10⁶ cfu/g, fermented at 50° C. for 3 days, and then fermented at 80° C. for 4 days.

The aged tea sample was pulverized for 15 seconds and then sieved using a stainless steel sieve having a mesh size of 1 mm. Thereafter, 50 mg of the pulverized tea sample was placed in a 1.5 ml Eppendorf tube, 1 ml of deionized water was added thereto, and the mixture was stirred at a constant speed for 30 minutes in a constant temperature water bath at 60° C. and then centrifuged at 25° C. and 13,000 rpm for 15 minutes. Only the portion which was not soluble in water was separated from the fermented green tea extract dried.

### [Example 2] Obtaining of Fraction and Separation of Compound

Catechin derivatives and caffeine were removed by fractionating 150 g of the post fermented tea sample using acetone, and a soluble in which other compounds were concentrated was obtained. By silica gel column chromatography, 40 g of the acetone soluble was first fractionated using a 5:1 (v/v) mixture of chloroform:methanol as a solvent.

By high-performance countercurrent chromatography (HPCCC, Dynamic Extractions Ltd, UK), 8.9 g of the caffeine-free 5:1 (v/v) fraction of chloroform:methanol was fractionated. The solvent used at this time was n-hexane-TBME (methyl tert-butyl ether)-BuOH-MeCN-Water (0.25:3:1:1:5, v/v), and the flow rate was set to 25 ml/min. Under the above conditions, 10 subfractions were obtained in total, and the components contained in each fraction were separated by small-capacity HPCCC (Dynamic Extractions Ltd, UK), HPLC (high-performance liquid chromatography), sephadex LH-20 column (GE Healthcare Bio-Sciences, Sweden), and the like.

As a result, it was possible to separate kaempferol3-O-[2-O″-(E)-p-coumaroyl][beta-D-glucopyranosyl-(1→3)-O-alpha-L-rhamnopyranosyl-(1→6)-O-beta-D-glucopyranoside], which was a compound unknown in the prior art, from the fractions, and the structure of each compound was identified by ¹H and ¹³C-NMR (nuclear magnetic resonance spectroscopy), UV (ultraviolet spectroscopy), and ESI-MS (electro spray ionization mass spectroscopy). In the case of ¹H and ¹³C nuclear magnetic resonance (NMR), methanol-d3 was used as a solvent and Bruker Advance DPX-500 (BRUKER, USA) was used as an instrument. The MS spectrum of each compound was attained using 6200 Series Accurate-Mass Time-of-Flight (TOF) LC/MS (Agilent, US).

As a result of the analysis, each of the above compounds has been confirmed to be kaempferol3-O-[2-O″-(E)-p-coumaroyl][beta-D-glucopyranosyl-(1→3)-O-alpha-L-rhamnopyranosyl-(1→6)-O-beta-D-glucopyranoside] which has a molecular formula of C₄₂H₄₆O₂₂ and a molecular weight of 902.2481 and is a novel compound unknown in the prior art.

The chemical formula and NMR data of kaempferol3-O-[2-O″-(E)-p-coumaroyl][beta-D-glucopyranosyl-(1→3)-O-alpha-L-rhamnopyranosyl-(1→6)-O-beta-D-glucopyranoside] are as follows.

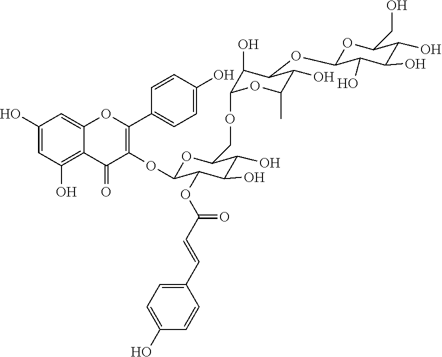 [SMILES: CC1OC(OCC2OC(Oc3c(-c4ccc(O)cc4)oc4cc(O)cc(O)c4c3=O)C(OC(=O)/C=C/c3ccc(O)cc3)C(O)C2O)C(O)C(OC2OC(CO)C(O)C(O)C2O)C1O]

TABLE 1

| Position | ¹³C-NMR | ¹H-NMR |
|---|---|---|
| 2 | 161.26 | |
| 3 | 135.07 | |
| 4 | 179.31 | |
| 5 | 161.5 | |
| 6 | 99.87 | 6.17 (H6, brs) |
| 7 | 165.74 | |
| 8 | 94.8 | 6.35 (H8, brs) |
| 9 | 158.58 | |
| 10 | 105.84 | |
| 1′ | 122.72 | |
| 2′, 6′ | 132.29 | 7.99 (H2′/H6′, d, |
| | | J = 8.3 Hz) |
| 3′, 5′ | 116.27 | 6.87 (H3′/H5′, |
| | | d, J = 8.3 Hz) |
| 4′ | 158.69 | |

p-coumaric acid

| | | |
|---|---|---|
| 1″′ | 127.3 | |
| 2″′, | 131.2 | 7.45 (H2″′/H6″′, |
| 6″′ | | d, J = 8.1 Hz) |
| 3″′, | 116.82 | 6.80 (H3″′/H5″′, |
| 5″′ | | d, J = 8.1 Hz) |
| 4″′ | 161.26 | |
| 7″′ | 115.31 | 6.35 (H7″′, d, |
| | | J = 15.7 Hz) |
| 8″′ | 146.88 | 7.67 (H8″′, d, |
| | | J = 15.7 Hz) |
| C═O | 168.79 | |
| Glc1 | | |
| 1″ | 101.55 | 5.46 (H1″, d, |
| | | J = 7.8 Hz) |
| 2″ | 74.14 | 5.34 (H2″, t, |
| | | J = 9 Hz) |
| 3″ | 73.25 | 3.76 (H3″, d, |
| | | J = 10.4 Hz) |
| 4″ | 70.47 | 3.85 (H4″, m) |
| 5″ | 75.51 | 3.73 (H5″, m) |
| 6″ | 67.54 | 3.76 (H6″, brd, |
| | | J = 10.4 Hz) |
| | | 3.54 (H6″, m) |

Rha

| | | |
|---|---|---|
| 1″″ | 101.85 | 4.60 (H1″″, brs) |
| 2″″ | 71.34 | 3.95 (2″″, m) |
| 3″″ | 83.09 | 3.61 (H3″″, dd, |
| | | J = 9, 3 |
| | | Hz) |
| 4″″ | 72.6 | 3.46 (H4″″, m) |
| 5″″ | 69.49 | 3.54 (H5″″, m) |
| 6″″ | 18.08 | 1.19 (H6″″, d, |
| | | J = 6 Hz) |
| 1″″′ | 105.74 | 4.40 (H1″″′, |
| | | d, J = 7.5 Hz) |
| 2″″′ | 75.4 | 3.25 (H2″″′, m) |
| 3″″′ | 77.6 | 3.36 (H3″″′, m) |
| 4″″′ | 70.84 | 3.36 (H4″″′, m) |
| 5″″′ | 77.6 | 3.25 (H5″″′, m) |
| 6″″′ | 62.05 | 3.71 (H6″″′, m) |

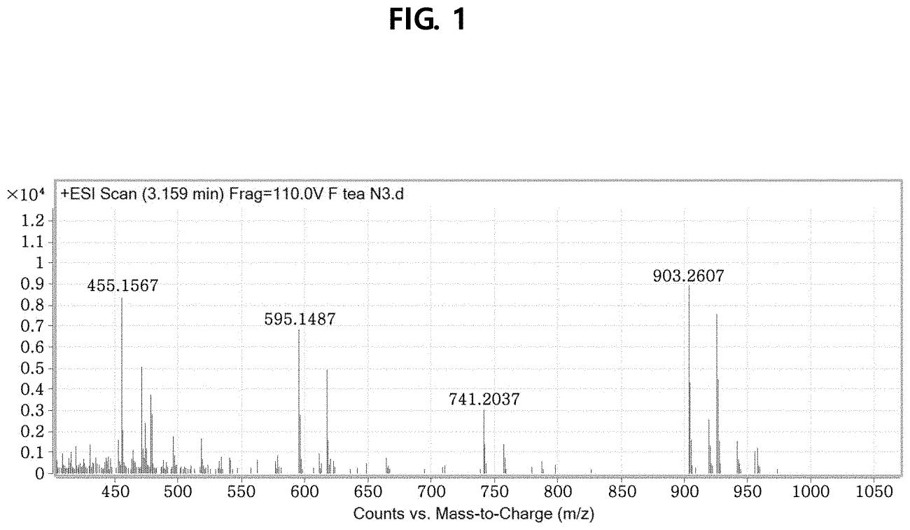

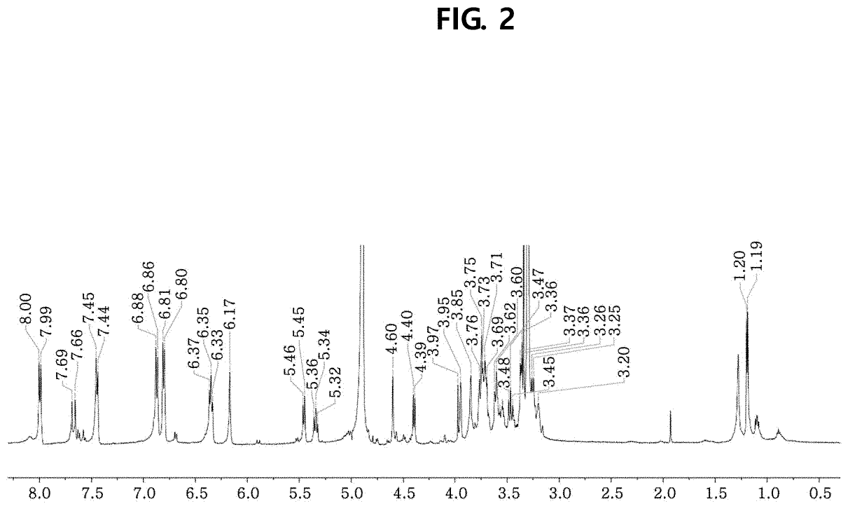

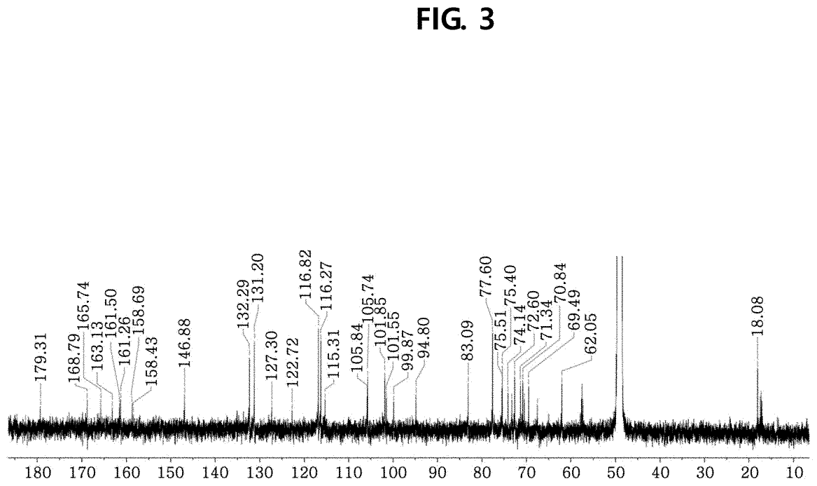

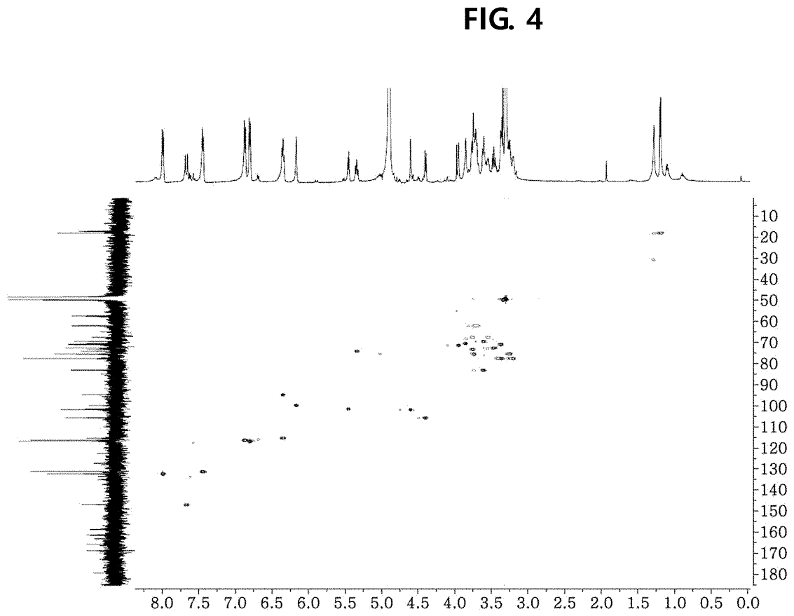

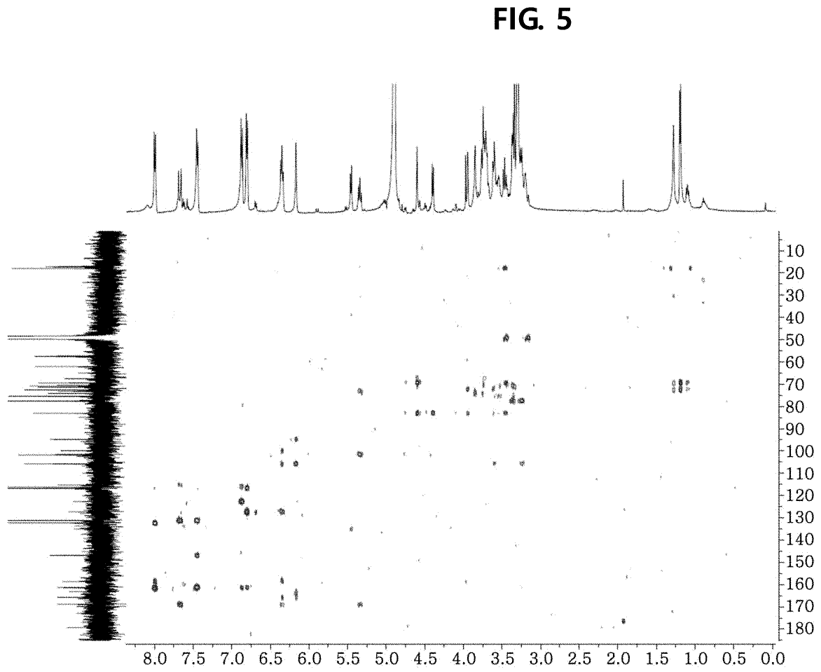

The MS spectrum of kaempferol3-O-[2-O″-(E)-p-coumaroyl][beta-D-glucopyranosyl-(1→3)-O-alpha-L-rhamnopyranosyl-(1→6)-O-beta-D-glucopyranoside] is as illustrated in FIG. 1, and the ¹H-NMR spectrum and ¹³C-NMR spectrum thereof are as illustrated in FIG. 2 and FIG. 3, respectively, the HSQC (heteronuclear single quantum coherence) spectrum thereof is as illustrated in FIG. 4, and the HMBC (heteronuclear multiple-bond coherence) spectrum thereof is as illustrated in FIG. 5.

### [Experimental Example 1] Experiment on Effect of Inhibiting Beta Amyloid Aggregation

The effect of kaempferol3-O-[2-O″-(E)-p-coumaroyl][beta-D-glucopyranosyl-(1→3)-O-alpha-L-rhamnopyranosyl-(1→6)-O-beta-D-glucopyranoside] on inhibition of beta amyloid aggregation was confirmed by the fluorescence analysis (ThioflavinT assay).

Specifically, beta amyloid (Aβ1-42, AnaSpec Inc., USA) was obtained and used at a concentration of 0.1 mg/ml and stored at −80° C. before use. Morin (20 μM), phenol red (20 μM), and kaempferol3-O-[2-O″-(E)-p-coumaroyl][beta-D-glucopyranosyl-(1→3)-O-alpha-L-rhamnopyranosyl-(1→6)-O-beta-D-glucopyranoside] (1 mg/ml) were respectively diluted with DMSO so as to be adjusted to the concentrations.

In order to specify the degree of inhibition of Aβ1-42 aggregation, each of the compounds prepared at the above concentrations was diluted with 50 μL of 0.01 M sodium phosphate buffer solution to have a concentration of 10 μM, then 40 μL of 0.1 mg/ml of Aβ1-42 was added thereto, and then 10 μl of 2 mM thioflavinT was added thereto, and fluorescence was measured using a fluorescence spectrometer (RF-5300PC, SHIMADZU CORPORATION, Japan) at 37° C. for 150 minutes at intervals of 5 minutes.

The results are as presented in the following Table 2 and FIG. 6.

TABLE 2

| | | Increased |
|---|---|---|
| | Increased | RFU (% of |
| | RFU | Pos. Cont.) |
| Pos. Cont. | 14595 | 100.0 |
| Novel substance 33 | 11235 | 77.0 |
| Morin | 11471 | 78.6 |
| Phenol Red | 13655 | 93.6 |

In the above table, “RFU” denotes the relative fluorescence unit, and “Increased RFU” denotes the amount of aggregated beta amyloid, and “Increased RFU (% of Pos.Cont.)” denotes the percentage value of the amount of aggregated beta amyloid with respect to that of the positive control group. “Novel substance 33” denotes kaempferol3-O-[2-O″-(E)-p-coumaroyl][beta-D-glucopyranosyl-(1→3)-O-alpha-L-rhamnopyranosyl-(1→6)-O-beta-D-glucopyranoside].

In other words, kaempferol3-O-[2-O″-(E)-p-coumaroyl][beta-D-glucopyranosyl-(1→3)-O-alpha-L-rhamnopyranosyl-(1→6)-O-beta-D-glucopyranoside] exhibited an effect of inhibiting the aggregation by 23.0% as compared with the positive control group when the aggregation in the positive control group (denoted by “Pos.Cont.”, only beta amyloid was aggregated without compound treatment) was taken as 100%. This result indicates that kaempferol3-O-[2-O″-(E)-p-coumaroyl][beta-D-glucopyranosyl-(1→3)-O-alpha-L-rhamnopyranosyl-(1→6)-O-beta-D-glucopyranoside] exhibits an effect of inhibiting aggregation and plaque formation of beta amyloid superior to those of morin (21.4%) and phenol red (6.4%) which are inhibitors known in the prior art. Consequently, the two compounds have the above-mentioned effects and can be thus used for prevention, treatment, and improvement of cognitive function decrease associated with β-amyloid aggregation. In addition, the compound can protect nerve cells from damage or death and prevent and inhibit damage or death of nerve cells, thereby protecting nerve cells, and this can realize protection of nerve cells.

### [Experimental Example 2] Cumulative Skin Irritation Experiment

Human repeated insult patch tests (HRIPT) were conducted to determine the cumulative skin irritation by kaempferol3-O-[2-O″-(E)-p-coumaroyl][beta-D-glucopyranosyl-(1→3)-O-alpha-L-rhamnopyranosyl-(1→6)-O-beta-D-glucopyranoside] and to calculate the concentration range in which kaempferol3-O-[2-O″-(E)-p-coumaroyl][beta-D-glucopyranosyl-(1→3)-O-alpha-L-rhamnopyranosyl-(1→6)-O-beta-D-glucopyranoside] can be used on the skin.

Specifically, 15 healthy adult subjects were randomly selected, the test compositions (compositions for skin containing an emulsifier, a stabilizer, purified water, and the like in addition to the compound) containing the compound at 0.5 wt %, 1 wt %, and 3 wt % were dropped by 20 μl per chamber (IQ chamber, Epitest Ltd, Finland), and the patch was applied to the right side of the upper back of the subject and then replaced with new one after 24 hours. The skin reaction was examined before and after the patch test while the patch test was conducted three times a week and thus nine times for three weeks in total in this manner, the skin reaction was observed until the 48th hour after removal of the final patch, and the average reactivity was determined.

The results are as presented in the following Table 3.

TABLE 3

| | Number of subjects showed ±, +, or ++ | | | | | | | | | |
|---|---|---|---|---|---|---|---|---|---|---|
| | reactivity (unit: persons) | | | | | | | | | |
| Test substance | 1st | 2nd | 3rd | 4th | 5th | 6th | 7 th | 8 th | 9th | Average |
| and content | time | time | time | time | time | time | time | time | time | reactivity |
| Control | 0 | 0 | 0 | 0 | 0 | 0 | 0 | 0 | 0 | 0 |
| group | 0 | 0 | 0 | 0 | 0 | 0 | 0 | 0 | 0 | |
| | 0 | 0 | 0 | 0 | 0 | 0 | 0 | 0 | 0 | |
| Novel | 0 | 0 | 0 | 0 | 0 | 0 | 0 | 0 | 0 | 0 |
| substance | 0 | 0 | 0 | 0 | 0 | 0 | 0 | 0 | 0 | |
| 33 at | 0 | 0 | 0 | 0 | 0 | 0 | 0 | 0 | 0 | |
| 0.5 wt % | | | | | | | | | | |
| Novel | 0 | 0 | 0 | 0 | 0 | 0 | 0 | 0 | 0 | 0 |
| substance | 0 | 0 | 0 | 0 | 0 | 0 | 0 | 0 | 0 | |
| 33 at | 0 | 0 | 0 | 0 | 0 | 0 | 0 | 0 | 0 | |
| 1 wt % | | | | | | | | | | |
| Novel | 0 | 0 | 0 | 0 | 0 | 0 | 0 | 0 | 0 | 0 |
| substance | 0 | 0 | 0 | 0 | 0 | 0 | 0 | 0 | 0 | |
| 33 at | 0 | 0 | 0 | 0 | 0 | 0 | 0 | 0 | 0 | |
| 3 wt % | | | | | | | | | | |

Reactivity
−: Negative (no reaction)
±: Suspicious or mild erythema and the like
+: Weak reaction (not accompanied by small vesicula), erythema, papule
++: Moderate reaction (with small vesicula), erythema, papule, vesicula
+++: Strong reaction, bulla reaction
Equation of average reactivity
Average reactivity = [{(sum of values obtained by multiplying the number of subjects who showed reactivity and reaction index)/(total number of subjects × highest score (4 points))} × 100]/Number of tests (9 times)
In the above equation, the reaction index is 0 when the reactivity is −, the reaction index is 1 when the reactivity is ±, the reaction index is 2 when the reactivity is +, the reaction index is 4 when the reactivity is ++.
It is judged as a safe composition when the average reactivity is less than 3.

The skin reaction was judged according to the criteria of the International Contact Dermatitis Research Group (ICDRG). In the above table, “Novel substance 33” denotes kaempferol3-O-[2-O″-(E)-p-coumaroyl][beta-D-glucopyranosyl-(1→3)-O-alpha-L-rhamnopyranosyl-(1→6)-O-beta-D-glucopyranoside]. In other words, the substance exhibited (−) reactivity (no subjects showed ±, +, ++, or +++ reactivity) in all the content ranges. Hence, it can be seen that the substance can be used safely on the skin without cumulative skin irritation.

### [Experimental Example 3] Confirmation of Expression Level of Intracellular BDNF (Brain-Derived Neurotrophic Factor) and DNMT1 (DNA (Cytosine-5)-Methyltransferase 1)

It was confirmed whether the novel substance 33 exerted its effect in cells as well.

Specifically, SH-SY5Y (neuroblastoma, Korean Cell Line Bank) cell line was seeded in a 6-well plate (FALCON) at 2×10⁶ cells per well, cultured in an incubator containing 5% CO₂ at 37° C. for 24 hours, then treated with 10 μg/ml of GCG, 10 μM of EGCG, 10 μg/ml of the existing green tea extract (GTE), 10 μg/ml of the novel substance 33, and 1 μM of 5-Aza-2′deoxycytidine (5-Aza, Sigma-aldrich) as a positive control group, and further cultured for 24 hours. Thereafter, the medium was entirely removed therefrom, and RNA was extracted therefrom using an RNA extraction kit (RNeasy mini kit, Quiagen). The extracted mRNA was quantitated using an ultraviolet detector (TECAN), and then 1 μg of mRNA was synthesized into a complementary DNA using a kit (SuperScript VILO cDNA Synthesis Kit, Thermofisher Scientific). Approximately 1 μg of the complementary DNA was taken and subjected to the real-time quantitative chain reaction using Taqman probe (Life technology) and Quantitect Probe PCR Kit (Quiagen). The expression levels of BDNF and DNMT1 were thus confirmed. At this time, the housekeeping gene, GAPDH, was used as the reference mRNA.

The expression levels of BDNF and DNMT1 are presented in Table 5 and Table 6, respectively.

TABLE 4
Relative expression level of BDNF

| Division | % |
|---|---|
| Control group (untreated group) | 100 |
| Novel substance 33 (10 μg/ml) | 128 |
| 5-Aza-2′deoxycytidine 1 μM | 149 |

TABLE 5
Relative expression level of DNMT1

| Division | % |
|---|---|
| Control group (untreated group) | 100 |
| Novel substance 33 (10 μg/ml) | 78 |
| 5-Aza-2′deoxycytidine 1 μM | 65 |

The novel substance 33 reduces DNMT1 expression and enhances BDNF expression, thus the compound can protect nerve cells from damage or death and prevent and inhibit damage or death of nerve cells, and this can realize protection of nerve cells and prevention and improvement of degenerative neurologic diseases.

Hereinafter, Formulation Examples of the composition according to an aspect of the present invention will be described, but the scope of the present invention is not limited thereto.

### [Formulation Example 1] Soft Capsule

A soft capsule was prepared by mixing 20 mg of kaempferol3-O-[2-O″-(E)-p-coumaroyl][beta-D-glucopyranosyl-(1→3)-O-alpha-L-rhamnopyranosyl-(1→6)-O-beta-D-glucopyranoside], 80 to 140 mg of L-carnitine, 180 mg of soybean oil, 2 mg of palm oil, 8 mg of hardened vegetable oil, 4 mg of yellow wax, and 6 mg of lecithin and filling the mixture in one capsule according to a conventional method.

### [Formulation Example 2] Tablet

A tablet was prepared by mixing 30 mg of kaempferol3-O-[2-O″-(E)-p-coumaroyl][beta-D-glucopyranosyl-(1→3)-O-alpha-L-rhamnopyranosyl-(1→6)-O-beta-D-glucopyranoside], 200 mg of galactooligosaccharide, 60 mg of lactose, and 140 mg of maltose, granulating the mixture using a fluidized bed dryer then adding 6 mg of sugar ester to the granules, and tableting the granules using a tablet machine.

### [Formulation Example 3] Granule

Granules were prepared by mixing 50 mg of kaempferol3-O-[2-O″-(E)-p-coumaroyl][beta-D-glucopyranosyl-(1→3)-O-alpha-L-rhamnopyranosyl-(1→6)-O-beta-D-glucopyranoside], 250 mg of anhydrous crystalline glucose, and 550 mg of starch, molding the mixture into granules using a fluidized bed granulator, and filling the granules in a bag.

### [Formulation Example 4] Drink

After 20 mg of kaempferol3-O-[2-O″-(E)-p-coumaroyl][beta-D-glucopyranosyl-(1→3)-O-alpha-L-rhamnopyranosyl-(1→6)-O-beta-D-glucopyranoside], 10 g of glucose, 0.6 g of citric acid, and 25 g of liquid oligosaccharide were mixed, 300 ml of purified water was added thereto, and the solution was filled in each bottle by 200 ml. The solution filled in the bottle was sterilized at 130° C. for from 4 to 5 seconds, thereby preparing a drink.

### [Formulation Example 5] Injection

An injection was prepared by a conventional method using 50 mg of kaempferol3-O-[2-O″-(E)-p-coumaroyl][beta-D-glucopyranosyl-(1→3)-O-alpha-L-rhamnopyranosyl-(1→6)-O-beta-D-glucopyranoside], suitable amount of sterile distilled water, and suitable amount of pH adjuster.

### [Formulation Example 6] Health Food

Health food was prepared by a conventional method according to the composition presented in the following Table 6.

TABLE 6

| Ingredient | Content | |
|---|---|---|
| Kaempferol3-O-[2-O″- | 0.5 | mg |
| (E)-p-coumaroyl][beta-D- | | |
| glucopyranosyl-(1→3)-O- | | |
| alpha-L-rhamnopyranosyl- | | |
| (1→6)-O-beta-D- | | |
| glucopyranoside] | | |

Vitamin mixture

| | | |
|---|---|---|
| Vitamin A acetate | 70 | μg |
| Vitamin E | 1.0 | mg |
| Vitamin B1 | 0.13 | mg |
| Vitamin B2 | 0.15 | mg |
| Vitamin B6 | 0.5 | mg |
| Vitamin B12 | 0.2 | μg |
| Vitamin C | 10 | mg |
| Biotin | 10 | μg |
| Nicotinic acid amide | 1.7 | mg |
| Folic acid | 50 | μg |
| Calcium pantothenate | 0.5 | mg |

Mineral mixture

| | | |
|---|---|---|
| Ferrous sulfate | 1.75 | mg |
| Zinc oxide | 0.82 | mg |
| Magnesium carbonate | 25.3 | mg |
| Potassium dihydrogen phosphate | 15 | mg |
| calcium monohydrogen phosphate | 55 | mg |
| Potassium citrate | 90 | mg |
| Calcium carbonate | 100 | mg |
| Magnesium chloride | 24.8 | mg |

The vitamin and mineral mixtures were prepared by mixing, for example, ingredients relatively suitable for health food but the compounding ratios thereof may be arbitrarily modified. The ingredients may be mixed according to a conventional method for manufacturing health food and then used in the preparation of a health food composition according to a conventional method.

### [Formulation Example 7] Health Drink

TABLE 7

| Ingredient | Content | |
|---|---|---|
| Kaempferol3-0-[2-O″- | 10 | mg |
| (E)-p-coumaroyl][beta-D- | | |
| glucopyranosyl-(1→3)-O- | | |
| alpha-L-rhamnopyranosyl- | | |
| (1→6)-O-beta-D- | | |
| glucopyranoside] | | |
| Citric acid | 1000 | mg |
| Oligosaccharide | 100 | g |
| Plum concentrate | 2 | g |
| Taurine | 1 | g |
| Purified water | Balance | |
| Total volume | 900 | ml |

As presented in Table 7, purified water was added as the balance so as to have a total volume of 900 ml, the ingredients were mixed according to a conventional method for manufacturing a health drink, the mixture was stirred and heated at 85° C. for about 1 hour. The solution thus prepared was filtered, filled in a sterilized 2-liter vessel, hermitically sealed, sterilized, and then stored in a refrigerator, thereby preparing a health drink.

Specific embodiments of the present specification have been described in detail above, and it will be apparent to those skilled in the art that this specific description is only preferred embodiments and that the scope of the present specification is not limited thereto. Accordingly, the actual scope of the present specification will be defined by the appended claims and their equivalents.

1. A method for improving cognitive function decrease comprising administering an effective amount of a compound represented by the following Chemical Formula 1, an isomer thereof, a pharmaceutically acceptable salt thereof, a hydrate thereof, a solvate thereof or a post fermented tea extract containing the compound, in need thereof: 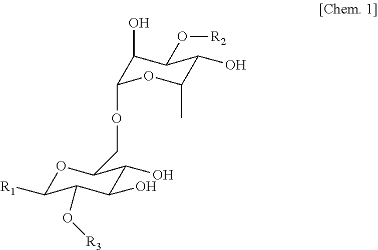
wherein R₁ represents C₁₅H₉O₆, R₂ represents C₆H₁₁O₅, and R₃ represents C₉H₇O₂.

2. The method according to claim 1, wherein R₁ denotes a compound represented by the following Chemical Formula 2:

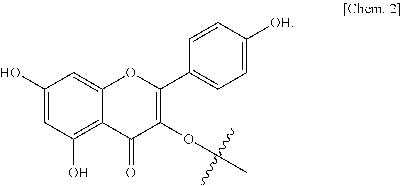 [SMILES: *Oc1c(-c2ccc(O)cc2)oc2cc(O)cc(O)c2c1=O]

3. The method according to claim 1, wherein R₂ denotes a compound represented by the following Chemical Formula 3:

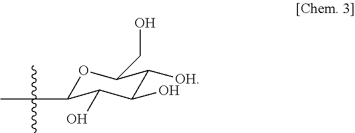 [SMILES: *C1OC(CO)C(O)C(O)C1O]

4. The method according to claim 1, wherein R₃ denotes a compound represented by the following Chemical Formula 4:

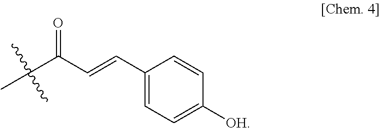 [SMILES: *C(=O)/C=C/c1ccc(O)cc1]

5. The method according to claim 1, wherein the compound is kaempferol3-O -[2-O″-(E)-p-coumaroyl][beta-D-glucopyranosyl-(1→3)-O-alpha-L-rhamnopyranosyl-(1→6)-O-beta-D-glucopyranoside].

6. The method according to claim 1, wherein the extraction is extraction using one or more solvents selected from hot water, a C₁ to C₆ lower alcohol, or any mixed solvent of the hot water and the C₁ to C₆ lower alcohol.

7. The method according to claim 6, wherein the lower alcohol is ethanol.

8. The method according to claim 1, wherein the extract is a fraction fractionated using a ketone after extraction.

9. The method according to claim 8, wherein the ketone is acetone.

10. The method according to claim 1,
wherein the compound represented by Chemical Formula 1, an isomer thereof, a pharmaceutically acceptable salt thereof, a hydrate thereof or a solvate thereof is administered in a form of a composition, wherein a content of the compound represented by Chemical Formula 1, an isomer thereof, a pharmaceutically acceptable salt thereof, a hydrate thereof or a solvate thereof in the composition is from 0.00001 wt % to 10 wt % based on a total weight of the composition.

11. The method according to claim 1,
wherein the compound represented by Chemical Formula 1, an isomer thereof, a pharmaceutically acceptable salt thereof, a hydrate thereof or a solvate thereof is administered in a form of a composition, wherein a content of the post fermented tea extract in the composition is from 0.1 wt % to 90 wt % based on a total weight of the composition.

12. The method according to claim 1, wherein the extract contains the compound represented by Chemical Formula 1, an isomer thereof, a pharmaceutically acceptable salt thereof, a hydrate thereof or a solvate thereof at from 0.0001 wt % to 20 wt % based on a total weight of the extract.

13. The method according to claim 1, wherein a dosage of the compound represented by Chemical Formula 1, an isomer thereof, a pharmaceutically acceptable salt thereof, a hydrate thereof or a solvate thereof by administration of the composition is from 0.001 mg/kg/day to 100 mg/kg/day.

14. The method according to claim 1, wherein the cognitive function decrease is caused by one or more selected from the group consisting of aggregation of β-amyloid, reduced expression of brain-derived neurotrophic factor (BDNF), and enhanced expression of DNMT1 (DNA (cytosine-5)-methyltransferase 1).

15. The method according to claim 1, wherein the cognitive function decrease includes one or more selected from the group consisting of memory loss, cognition decline, discrimination decline, depression, and forgetfulness.

16. The method according to claim 1, wherein the improvement is achieved through one or more selected from the group consisting of inhibition of β-amyloid aggregation, degradation of aggregated β-amyloid, enhancement of BDNF expression, and reduction of DNMT1 expression.

17. The method according to claim 1, wherein the composition is fora nerve cell protection.

18. The method according to claim 17, wherein the nerve cell protection is to protect a nerve cell from influence of one or more selected from the group consisting of aggregation of β-amyloid, reduced expression of brain-derived neurotrophic factor (BDNF), and enhanced expression of DNMT1 (DNA (cytosine-5)-methyltransferase 1).

19. The method according to claim 1,
wherein the compound represented by Chemical Formula 1, an isomer thereof, a pharmaceutically acceptable salt thereof, a hydrate thereof or a solvate thereof is administered in a form of a composition, wherein the composition is a food or pharmaceutical composition.
